(19) United States
(12) Patent Application Publication
(10) Pub. No.: US 2018/0364196 A1
(43) Pub. Date: Dec. 20, 2018

(54) METHOD FOR IMPROVING THE ACCURACY OF OXYGEN CONCENTRATION DETECTION

(71) Applicant: Foshan Keyhub Electronic Industries CO., Ltd, Foshan (CN)

(72) Inventors: Jack ONG, Foshan (CN); Leon ZHANG, Foshan (CN); Bill DENG, Foshan (CN)

(21) Appl. No.: 16/061,576

(22) PCT Filed: Nov. 12, 2016
(86) PCT No.: PCT/CN2016/105544

§ 371 Date: Jun. 12, 2018

(30) Foreign Application Priority Data
Nov. 11, 2016 (CN) 201610991521.7

(51) Int. Cl. G01N 29/024; G01N 29/32; G01N 33/00

(52) U.S. Cl. CPC G01N 29/024; G01N 2291/021; G01N 33/0036; G01N 29/326

(57) ABSTRACT

A method for improving the accuracy of oxygen concentration detection comprises the following steps: in a first step, when a gas to be detected enters a gas tube, the gas tube is connected to a detection channel and the gas enters the detection channel, the detection channel being a relatively-fixed sealed space only provided with a gas inlet and outlet; in a second step, initiate an ultrasonic wave generator located at one end of the detection channel, and initiate an ultrasonic wave receiver at the other end of the detection channel; and in a third step, in a fixed time segment ranging from 0.001 s to 0.01 s, a control chip records accurate reception time in which an ultrasonic sensor sends a startup to the ultrasonic wave receiver, and calculates the oxygen concentration in the time segment by using an calculation formula.

### TECHNICAL FIELD OF THE INVENTION

The present invention relates to the technical field of detecting medical oxygen concentration, and more particularly, to a method for improving the accuracy of oxygen concentration detection.

### BACKGROUND OF THE INVENTION

In the prior art, the traditional oxygen concentration detectors usually detect the proportion of oxygen in the nitrogen-oxygen gas mixture by using the ultrasonic technology, wherein the calculation formula of the ultrasonic propagation velocity V is the following:

V=√⁻(γ*R*T/M)

During the sampling process, the sampling data may fluctuate greatly along the variation of the flowing gases, resulting in a poor sampling consistency of the ultrasonic propagation velocity V. Moreover, as the gas inlet A and the gas outlet B of the sample gas channel form a certain angle with the detection channel, the gas flow velocity and the gas flow direction at the two ends are inconsistent with that in the whole detection channel. Thus, detection errors can easily occur. Furthermore, in order to eliminate the impact caused by the gas flow variation at the two ends, a longer detection channel is required for satisfying the detection requirement, leading to a high material cost.

### SUMMARY OF THE INVENTION

The purpose of the present invention is to solve the shortcomings in the prior art by providing a method for improving the accuracy of oxygen concentration detection, which is simple and convenient for users.

To achieve the above purpose, the present invention adopts the following technical solution:

A method for improving the accuracy of oxygen concentration detection, comprising the steps of:

Step 1: introducing the gas to be detected into a gas tube; the gas tube being connected with a detection channel, thereby enabling the gas to be detected to simultaneously enter into the detection channel; the detection channel being a relatively-fixed sealed space only provided with a gas inlet and outlet;

Step 2: initiating an ultrasonic wave generator located at one end of the detection channel, and initiating an ultrasonic wave receiver at the other end of the detection channel;

Step 3: in a fixed-time segment ranging from 0.001 s to 0.01 s, recording accurate reception time in which an ultrasonic sensor sends a startup to the ultrasonic wave receiver, and calculating the oxygen concentration in the time segment by using a calculation formula via a control chip;

In another preferred embodiment, after step 1 is completed, the detection channel is in a static state, and the gas tube is in a gas-flowing state. The gas tube and the detection channel do not interfere with each other.

In another preferred embodiment, after step 1 is completed, the high-concentration gas is diffused to the low-concentration gas, and the gas concentrations in the sample gas channel and the detection channel reach a dynamic balance. In a single sampling period (0.01 s), the oxygen partial pressure in the detection channel remains stable.

In another preferred embodiment, in the method of the present invention, the gas to be detected does not interfere with the ultrasonic detection device.

In another preferred embodiment, according to the method of the present invention, the oxygen concentration a can be calculated by using the following calculation formula 1 after the molar mass of the gas mixture M is measured out.

M=MO2*a%+MN2*(1−a%) □

The ultrasonic propagation velocity V can be calculated by using the following calculation formula 2, wherein γ is the specific heat ratio of the gas mixture, R is a gas constant that is equal to 8.31, T is the gas temperature and M is the molar mass of the gas mixture.

V=√⁻(γ*R*T/M) □

After the ultrasonic propagation velocity V is obtained, the ultrasonic propagation distance L can be calculated through the following calculation formula 3, wherein t is the ultrasonic propagation time.

V=L/t \textcircled{3}

The time error from sending a startup by the ultrasonic sensor to the accurate reception is defined as ^{Δ}t, which can be calculated through the following calculation formula 4, wherein t is the actual propagation time measured by the control system.

V=L/(t−Δt) □

According to the foresaid formula 2, when the molar mass of the gas mixture M1 and M2 and the gas temperature T1 and T2 are known, the ultrasonic propagation velocity V1 and V2 in two different temperature states can be calculated. After the ultrasonic propagation velocity V1 and V2 are substituted into the following two formulas, the values of L and ^{Δ}t can be obtained.

V1=L/(t1−^{Δ}t) V2=L/(t2−^{Δ}t)

When the gas concentration is unknown, the ultrasonic propagation velocity V can be calculated by using aforesaid formula 4 after L and ^{Δ}t are calculated out and the gas temperature T is obtained through actual measurement, and the oxygen concentration a can be calculated through the aforesaid formulas 2 and 1.

Compared with the prior art, the present invention has the following advantages:

According to the aforesaid technical solution, the oxygen concentration can be calculated through an embedded computing center. As the gas to be detected does not interfere with the ultrasonic detection device, the three values including V, ^{Δ}t, and L can be precisely calculated. Thus, the accuracy of oxygen concentration detection can be greatly improved. Furthermore, it's unnecessary to use a longer detection channel so that the material cost can be saved. Due to the reduced size of the detection device, the oxygenator can be miniaturized.

### BRIEF DESCRIPTION OF THE DRAWINGS

To clearly expound the technical solution of the present invention, the drawings and embodiments are hereinafter combined to illustrate the present invention. Obviously, the drawings are merely some embodiments of the present invention and those skilled in the art can associate themselves with other drawings without paying creative labor.

FIG. 1 is a principle diagram of the prior art;

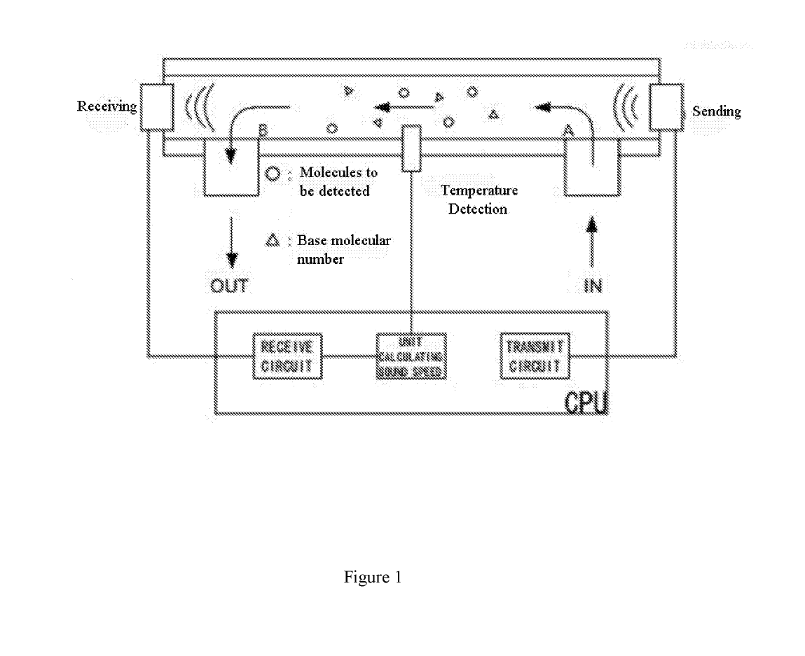

FIG. 2 is a principle diagram of the method for improving the accuracy of oxygen concentration detection of the present invention, wherein the circular molecule is molecule to be detected, and the triangular molecule is base molecular number.

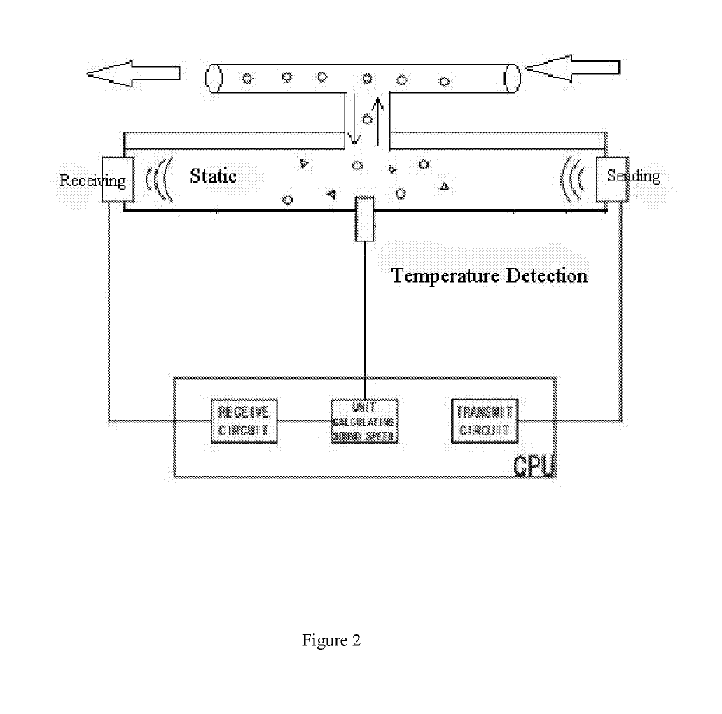

### DETAILED DESCRIPTION OF THE INVENTION

Drawings and detailed embodiments are combined hereinafter to elaborate the technical principles of the present invention.

A method for improving the accuracy of oxygen concentration detection, comprising the steps of:

Step 1: introducing the gas to be detected into a gas tube; the gas tube being connected with a detection channel, thereby enabling the gas to be detected to simultaneously enter into the detection channel; the detection channel being a relatively-fixed sealed space only provided with a gas inlet and outlet;

Step 2: initiating an ultrasonic wave generator located at one end of the detection channel, and initiating an ultrasonic wave receiver at the other end of the detection channel;

Step 3: in a fixed-time segment ranging from 0.001 s to 0.01 s, recording accurate reception time in which an ultrasonic sensor sends a startup to the ultrasonic wave receiver, and calculating the oxygen concentration in the time segment by using a calculation formula via a control chip;

After step 1 is completed, the detection channel is in a static state, and the gas tube is in a gas-flowing state. The gas tube and the detection channel do not interfere with each other.

After step 1 is completed, the high-concentration gas is diffused to the low-concentration gas, and the gas concentrations in the sample gas channel and the detection channel reach a dynamic balance. In a single sampling period (0.01 s), the oxygen partial pressure in the detection channel can be regarded as unchanged.

In the method of the present invention, the gas to be detected does not interfere with the ultrasonic detection device.

According to the method of the present invention, the oxygen concentration a can be calculated by using the following calculation formula 1 after the molar mass of the gas mixture M is measured out.

M=MO2*a%+MN2*(1−a%) □

The ultrasonic propagation velocity V can be calculated by using the following calculation formula 2, wherein γ is the specific heat ratio of the gas mixture, R is a gas constant that is equal to 8.31, T is the gas temperature and M is the molar mass of the gas mixture.

V=√⁻(γ*R*T/M) □

After the ultrasonic propagation velocity V is obtained, the ultrasonic propagation distance L can be calculated through the following calculation formula 3, wherein t is the ultrasonic propagation time.

V=L/t \textcircled{3}

The time error from sending a startup by the ultrasonic sensor to the accurate reception is defined as ^{Δ}t, which can be calculated through the following calculation formula 4, wherein t is the actual propagation time measured by the control system.

V=L/(t−Δt) □

According to the foresaid formula 2, when the molar mass of the gas mixture M1 and M2 and the gas temperature T1 and T2 are known, the ultrasonic propagation velocity V1 and V2 in two different temperature states can be calculated. After the ultrasonic propagation velocity V1 and V2 are substituted into the following two formulas, the values of L and ^{Δ}t can be obtained.

V1=L/(t1−^{Δ}t) V2=L/(t2−^{Δ}t)

When the gas concentration is unknown, the ultrasonic propagation velocity V can be calculated by using aforesaid formula 4 after L and ^{Δ}t are calculated out and the gas temperature T is obtained through actual measurement, and the oxygen concentration a can be calculated through the aforesaid formulas 2 and 1.

The present invention utilizes the principle of gas diffusion. In the present invention, the gas to be detected and the detection channel respectively stay in a dynamic state and a static state. As the ultrasonic detection channel is in a static state, the high-concentration gas is diffused to the low-concentration gas according to Fick's Law. Finally, the gas concentrations in the sample gas channel and the detection channel reach a dynamic balance. In a single sampling period (0.001 s-0.01 s), the oxygen partial pressure in the detection channel can be regarded as unchanged. Based on these conditions, the sampling requirement can be satisfied, and the technical problems in the prior art can be solved.

The operating principle of the present invention is the following:

The ultrasonic waves carry the information of the flow velocity of the fluid when propagating in a flowing fluid. Thus, the flow velocity of the fluid can be detected according to the received ultrasonic waves, and can be further converted into a flow quantity. The ultrasonic pulses pass through the gas tube, and arrive at the other sensor from one sensor. This process resembles a boatman sailing across a river. When the gas no longer flows, the acoustic pulses are propagated in two directions at the same speed (acoustic velocity, C). When the gas in the gas tube possesses a certain flow velocity V (the flow velocity is not equal to zero), the acoustic pulses in the flow direction can be rapidly propagated, and those in the reverse direction can be propagated slowly. In this way, the down-current propagation time tD is shorter, and the counter-current propagation time tU is longer.

The shorter or longer propagation time described above is compared to the propagation time when the gas does not flow. According to different detection modes, the detection method can be roughly divided into a propagation velocity-difference method, a Doppler method, a beam deviation method, a noise method and a correlation method, etc. The ultrasonic flow meters have been used in recent years along with the rapid development of integrated circuit technology.

According to different detection principles, the ultrasonic flow meter detection method can be generally divided into a propagation velocity difference method (including a direct time difference method, a time difference method, a phase difference method and a frequency difference method), a beam deviation method, a Doppler method, a correlation method, a spatial filtering method and a noise method, etc. Among them, the ultrasonic flow meter utilizing the noise detection principle has a simple structure and a low cost, and can be conveniently used and carried. However, as the detection accuracy is low, it's only suitable for occasions with low accuracy requirement.

The direct time difference method, the time difference method, the frequency difference method and the phase difference method are also called propagation velocity difference method. Their basic principle is to reflect the flow velocity of the fluid through detecting the velocity difference between the down-current propagation and the counter-current propagation of the ultrasonic pulses. The frequency difference method and the time difference method are widely used because they have a high accuracy, and are capable of overcoming the errors caused by the acoustic velocity that varies along with the variation of the fluid temperature. According to different configuration methods of the energy converter, the propagation velocity difference method can also be divided into a Z method (penetrant method), a V-method (reflection method) and an X-method (crossing method), etc.

The beam deviation method reflects the flow velocity of the fluid by using the propagation direction of the ultrasonic wave beam in the fluid that deviates along the variation of the flow velocity of the fluid. When the flow velocity is low, its sensitivity significantly decreases, resulting in a low applicability.

The Doppler method utilizes the acoustic Doppler principle to determine the flow quantity of the fluid through measuring the ultrasonic Doppler shift scattered by the scattering objects in a non-uniform fluid. This method is suitable for measuring the flow quantity of suspension particles or bubbles.

The correlation method utilizes correlation technique to measure the flow quantity of the fluid. As the detection accuracy of this method is irrelevant to the acoustic velocity of the fluid, it is also irrelevant to the fluid temperature and concentration. Therefore, the correlation method has a high accuracy and a wide application range. However, the correlator is expensive, and its circuit structure is relatively complex. The aforesaid shortcomings cannot be overcome before the microprocessor is popularized.

The noise method (sounding method) detects the flow velocity or flow quantity of the fluid through detecting the noise. It utilizes the principle that the noise generated when fluid flows in a tube is related to the flow velocity of the fluid. This method is cheap but inaccurate.

The novelty of the present invention is that the gas to be detected can be kept staying in a static environment for a long time. Thus, the ultrasonic concentration detection can be facilitated, and the detection accuracy can be greatly improved. In addition, the gas to be detected can be prevented from polluting the previous sample gas.

Chinese patent 201210303712.1 (the publication no. is CN 102830164A and the filing date is Aug. 23, 2012) discloses a method and a device for online-detecting the concentration of methane.

The aforesaid patent (hereinafter referred to as “comparison file”) relates to a method for detecting the concentration of methane in air under a coal mine, wherein the detection tube 4 is equivalent to the gas tube of the present invention, and the static tube 5 is equivalent to the detection channel of the present invention. The structural difference between them is that the comparison file has two diffusion tubes 6 whereas the present invention has only one. As the detection object in the comparison file is air, the diameter of its detection tube is greater than that of the static tube. On contrary, in the present invention, the diameter of the gas tube is smaller than that of the detection channel. This design aims to provide enough space to the binary nitrogen-oxygen gas, thereby enabling it to stay in a static state for a long time. In the comparison file, there're two diffusion tubes 6, which inevitably leads to the gas flow in the static tube 5. The present invention has only one gas tube that is connected with the detection channel. As a result, the oxygen gas can be kept in a static state, and the detection accuracy can be greatly improved.

Moreover, the detection method of the comparison file needs to quickly detect whether a critical value is reached and whether an alarm needs to be given. Therefore, this detection method focuses on a rapid online detection, and obviously, the detection accuracy is low. Furthermore, the detection device of the comparison file is not designed for an accurate detection whereas the present invention is specially designed for accurately detecting the binary nitrogen-oxygen gas. The gas to be detected only contains nitrogen and oxygen. The present invention focuses on the detection accuracy, which is the major difference between the comparison file and the present invention.

The description of above embodiments allows those skilled in the art to realize or use the present invention. Without departing from the spirit and essence of the present invention, those skilled in the art can combine, change or modify correspondingly according to the present invention. Therefore, the protective range of the present invention should not be limited to the embodiments above but conform to the widest protective range which is consistent with the principles and innovative characteristics of the present invention. Although some special terms are used in the description of the present invention, the scope of the invention should not necessarily be limited by this description. The scope of the present invention is defined by the claims.

1. A method for improving the accuracy of oxygen concentration detection, comprising the steps of:
Step 1: introducing the gas to be detected into a gas tube; the gas tube being connected with a detection channel, thereby enabling the gas to be detected to simultaneously enter into the detection channel; the detection channel being a relatively-fixed sealed space only provided with a gas inlet and outlet;
Step 2: initiating an ultrasonic wave generator located at one end of the detection channel, and initiating an ultrasonic wave receiver at the other end of the detection channel;
Step 3: in a fixed time segment ranging from 0.001 s to 0.01 s, recording accurate reception time in which an ultrasonic sensor sends a startup to the ultrasonic wave receiver, and calculating the oxygen concentration in the time segment by using a calculation formula via a control chip;

2. The method for improving the accuracy of oxygen concentration detection of claim 1, wherein after step 1 is completed, the detection channel is in a static state, and the gas tube is in a gas-flowing state, wherein the gas tube and the detection channel do not interfere with each other.

3. The method for improving the accuracy of oxygen concentration detection of claim 1, wherein after step 1 is completed, the high-concentration gas is diffused to the low-concentration gas, and the gas concentrations in the sample gas channel and the detection channel reach a dynamic balance, wherein in a single sampling period (0.01 s), the oxygen partial pressure in the detection channel can be regarded as unchanged.

4. The method for improving the accuracy of oxygen concentration detection of claim 1, wherein in the method of the present invention, the gas to be detected does not interfere with the ultrasonic detection device.

5. The method for improving the accuracy of oxygen concentration detection of claim 1, wherein according to the method of the present invention, the oxygen concentration can be calculated by using the following calculation formula 1 after the molar mass of the gas mixture M is measured out,
M=MO2*a%+MN2*(1−a%) □,
wherein the ultrasonic propagation velocity V can be calculated by using the following calculation formula 2,
V=√⁻(γ*R*T/M) □,
wherein γ is the specific heat ratio of the gas mixture, R is a gas constant that is equal to 8.31, T is the gas temperature and M is the molar mass of the gas mixture, wherein after the ultrasonic propagation velocity V is obtained, the ultrasonic propagation distance L can be calculated through the following calculation formula 3, V=L/t--------\textcircled{3}, wherein t is the ultrasonic propagation time, wherein the time error from sending a startup by the ultrasonic sensor to the accurate reception is defined as ^{Δ}t, which can be calculated through the following calculation formula 4, V=L/(t−Δt)--------□, wherein t is the actual propagation time measured by the control system, wherein according to the foresaid formula 2, when the molar mass of the gas mixture M1 and M2 and the gas temperature T1 and T2 are known, the ultrasonic propagation velocity V1 and V2 in two different temperature states can be calculated, wherein after the ultrasonic propagation velocity V1 and V2 are substituted into the following two formulas, V1=L/(t1−^{Δ}t), V2=L/(t2−^{Δ}t), the values of L and ^{Δ}t can be obtained, wherein when the gas concentration is unknown, the ultrasonic propagation velocity V can be calculated by using aforesaid formula 4 after L and ^{Δ}t are calculated out and the gas temperature T is obtained through actual measurement, and the oxygen concentration a can be calculated through aforesaid formulas 2 and 1.
